(19) United States
(12) Patent Application Publication
(10) Pub. No.: US 2010/0081985 A1
(43) Pub. Date: Apr. 1, 2010

(54) Platelet Additive Solution For Leukoreducing White Blood Cells In Apheresed Platelets

(75) Inventors: John PITTINGER, Evergreen, CO (US); Anna RAZATOS, Littleton, CO (US)

Correspondence Address: CaridianBCT, Inc.;Mail Stop: 810 1F2, 10811 WEST COLLINS AVE, LAKEWOOD, CO, 80215, US

(73) Assignee: CARIDIANBCT, INC., Lakewood, CO (US)

(21) Appl. No.: 12/572,029

(22) Filed: Oct. 1, 2009

Related U.S. Application Data
(60) Provisional application No. 61/101,693, filed on Oct. 1, 2008.

(51) Int. Cl. A61M 1/38

(52) U.S. Cl. 604/6.03

(57) ABSTRACT

This invention relates to a method of reducing residual white blood cells in an apheresed platelet product. The method includes the steps of adding to the platelet product a solution comprising sodium chloride and magnesium and inducing degradation of the residual white blood cells.

### PRIORITY CLAIM

This application claims priority from U.S. Provisional Application No. 61/101,693, filed Oct. 1, 2008.

### BACKGROUND

Human blood contains a number of components, including plasma, platelets, and red blood cells. Blood also contains components such as various types of white blood cells, and proteins of the complement system, that provide for combating infection.

Blood components may be separated from each other, and further processed, for a variety of uses, particularly as transfusion products. Illustratively, red blood cells (typically concentrated as packed red blood cells), plasma, and platelets (typically concentrated as platelet concentrate), can be separately administered to different patients. Some components, e.g., plasma and/or platelets, can be pooled before administration, and plasma can be fractionated to provide enriched protein components to treat diseases.

Typically, donated platelets are separated from other blood components using a centrifuge. The centrifuge rotates whole blood to separate components including platelets using centrifugal force. In use, blood enters the centrifuge while it is rotating at a very rapid speed and centrifugal forces stratifies the blood components so that particular components may be separately removed according to their densities. Centrifuges are effective at separating platelets from whole blood, however, they are typically unable to separate all of the white blood cells from the platelets to produce a platelet product that meets the “leukopoor” standard of less than 5×10⁶ white blood cells for at least 3×10¹¹ platelets collected.

Because typical centrifuge platelet collection processes are unable to completely separate white blood cells from platelets, other processes have been added to improve results. In one procedure, after centrifugation, platelets are passed through a porous woven or non-woven media filter, which may have a modified surface, to remove white blood cells. However, use of the porous filter introduces its own set of problems. Conventional porous filters may be inefficient because they may permanently remove or trap approximately 5-20% of the platelets. These conventional filters may also reduce “platelet viability,” meaning that once passed through a filter a percentage of the platelets cease to function properly and may become partially or fully activated. In addition, porous filters may cause platelets to release bradykinin, which may lead to hypotensive episodes in a patient. Porous filters are also expensive and often require additional time consuming manual labor to perform a filtration process.

Another separation process known as centrifugal elutriation, separates cells suspended in a liquid medium without the use of a membrane filter. In one common form of elutriation, cells are introduced into a flow of liquid elutriation buffer. This liquid which carries the cells in suspension, is then introduced into a funnel-shaped chamber located in a spinning centrifuge. As additional liquid buffer solution flows through the chamber, the liquid sweeps smaller sized, slower-sedimenting cells toward an elutriation boundary within the chamber, while larger, faster-sedimenting cells migrate to an area of the chamber having the greatest centrifugal force.

When the centrifugal force and force generated by the fluid flow are balanced, the fluid flow is increased to force slower-sedimenting cells from an exit port in the chamber, while faster-sedimenting cells are retained in the chamber. If fluid flow through the chamber is increased, progressively larger, faster-sedimenting cells may be removed from the chamber.

Thus, centrifugal elutriation separates particles having different sedimentation velocities. Stoke's law describes sedimentation velocity (SV) of a spherical particle as follows:

$$sv = \frac{2}{9} \frac{{r}^{2} ( {ρ}_{p} - {ρ}_{m} ) g}{η}$$

where,
r is the radius of the particle,
ρₚ is the density of the particle,
ρₘ is the density of the liquid medium,
η is the viscosity of the medium; and
g is the gravitational or centrifugal acceleration.

Because the radius of a particle is raised to the second power in the Stoke's equation and the density of the particle is not, it is the size of a cell, rather than its density, which greatly influences its sedimentation rate. This explains why larger particles generally remain in a chamber during centrifugal elutriation, while smaller particles are released, if the particles have similar densities.

Another method of leukoreduction includes a process for forming a saturated fluidized particle bed as described in U.S. Pat. No. 5,939,319.

However, in all of these leukoreduction procedures, there is usually some small percentage of white blood cells which are not captured by one of the leukoreduction mediums, and are carried over into a separated platelet component.

Carryover of white blood cells into platelet products is undesirable because white blood cells may transmit infections to recipients of the platelet products such as HIV and CMV, and cause other transfusion-related complications such as transfusion-associated Graft vs. Host Disease (TA-GVHD), alloimmunization and microchimerism. White blood cells may also become activated during storage and release cytokines. Cytokine accumulation during storage of platelet concentrates may mediate nonhemolytic febrile transfusion reactions in the recipient.

Since the problem arises from the presence of white cells in the donated platelet products, an additional process to remove or kill the residual white blood cells from separated platelets would be desirable. It is to this additional process that the present invention is directed.

### SUMMARY OF THE INVENTION

This invention relates to a method of reducing residual white blood cells in an apheresed platelet product. The method includes the steps of adding to the platelet product a solution comprising sodium chloride and magnesium; and inducing degradation of the residual white blood cells. Degradation of the residual white blood cells caused by the addition of the solution reduces any residual white blood cells which may be contained in the platelet product.

This invention also relates to a method for further leukoreducing leukoreduced apheresed platelets suitable for transfusion into a patient. This method includes the steps of providing hyperconcentrated apheresed leukoreduced platelets and resuspending the platelets in a solution containing at least sodium chloride and magnesium and lacking citrate.

### BRIEF DESCRIPTION OF THE DRAWINGS

FIG. 1 is a perspective view of one embodiment of an apheresis system which can be used with the present invention.

FIG. 2A-B illustrate an extracorporeal tubing circuit and cassette assembly for the system of FIG. 1.

FIG. 3 is an exploded, perspective view of the channel assembly for the system of FIG. 1.

FIG. 4 is a top view of the channel housing from the channel assembly of FIG. 1 illustrating various dimensions.

FIG. 5 is a perspective view of the blood processing vessel of the channel assembly of FIG. 8 in a disassembled view.

FIG. 6 is a cross-sectional view of the blood processing vessel taken along lines 18-18 in FIG. 5.

FIG. 7 is a front view of a pump/valve/sensor assembly for the system of FIG. 1.

FIG. 8 is a partial schematic view of the apparatus of the blood processing vessel of FIG. 1 illustrating a detailed view of components of the apparatus.

FIG. 9 is a graph comparing rWBC from apheresed platelets in various storage solutions over time.

FIG. 10 is a graph comparing rWBC from separated whole blood in various storage solutions over time.

FIG. 11 is a graph comparing the permeability of the membranes of apheresed WBC.

FIG. 12 is a graph comparing Annexin V expression on platelets stored in a platelet additive solution containing moderate amounts of magnesium and plasma.

### DETAILED DESCRIPTION

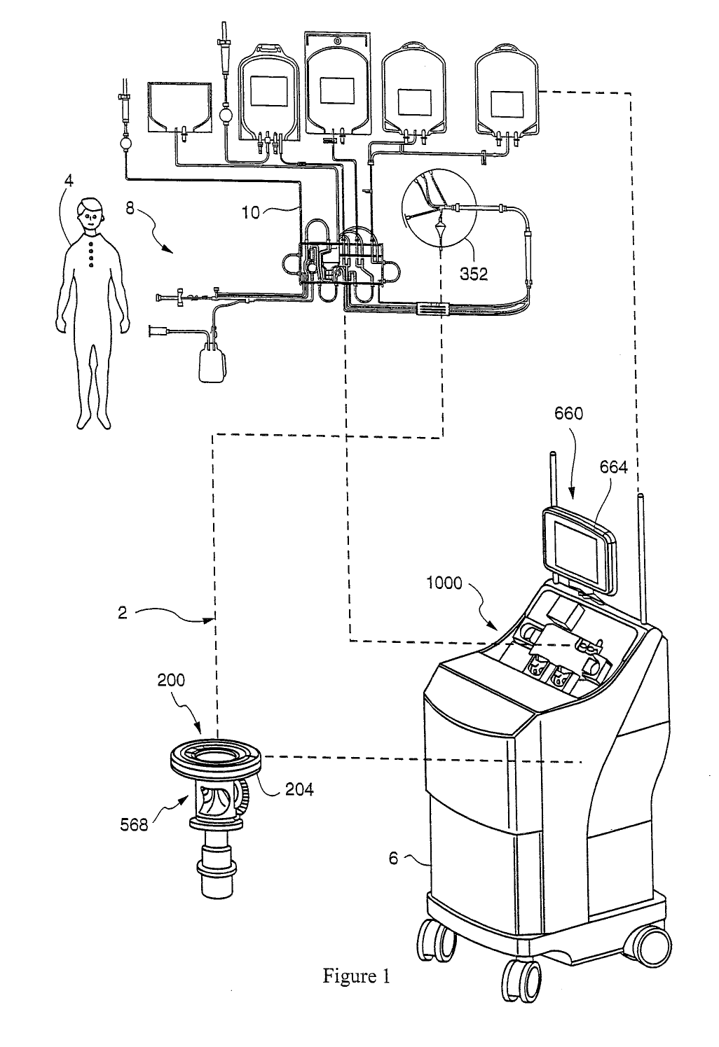

A blood apheresis system 2 for use in and/or with the present invention is schematically illustrated in FIG. 1. System 2 provides for a continuous blood component separation process. Generally, whole blood is withdrawn from a donor 4 and is substantially continuously provided to a blood component separation device 6 where the blood is continuously separated into various component types. One or more of the separated blood components may then either be collected for subsequent transfusion or may be uncollected and returned to the donor 4.

In the blood apheresis system 2, blood is withdrawn from the donor 4 and directed through a preconnected bag and tubing set 8 which includes an extracorporeal tubing circuit 10 and a blood processing vessel 352 which together define a closed, sterile and disposable system. The set 8 is adapted to be mounted on and/or in the blood component separation device 6. The separation device 6 preferably includes a pump/valve/sensor assembly 1000 for interfacing with the extracorporeal tubing circuit 10, and a channel assembly 200 for interfacing with the disposable blood processing vessel 352.

The channel assembly 200 may include a channel housing 204 which is rotatably interconnected with a rotatable centrifuge rotor assembly 568 which provides the centrifugal forces required to separate blood into its various blood component types by centrifugation. The blood processing vessel 352 may then be interfitted within the channel housing 204. When connected as described, blood can then be flowed substantially continuously from the donor 4, through the extracorporeal tubing circuit 10, and into the rotating blood processing vessel 352. The blood within the blood processing vessel 352 may then be continuously separated into various blood component types and at least one of these blood component types (e.g., platelets, plasma, or red blood cells) is continually removed from the blood processing vessel 352. Blood components which are not being retained for collection are also removed from the blood processing vessel 352 and returned to the donor 4 via the extracorporeal tubing circuit 10.

Operation of the blood component separation device 6 is controlled by one or more processors included therein, and may comprise a plurality of embedded computer processors to accommodate interface with ever-increasing PC user facilities (e.g., CD ROM, modem, audio, networking and other capabilities). Relatedly, in order to assist the operator of the apheresis system 2 with various aspects of its operation, the blood component separation device 6 includes a graphical interface 660 with an interactive touch screen 664.

A plurality of other known apheresis systems may also be useful herewith, as for example, the Baxter CS3000 and/or AMICUS and/or AUTOPHERESIS-C systems, and/or the Haemonetics MCS or MCS+ and/or the Fresenius COM.TEC or AS-104 and/or the CaridianBCT TRIMA ACCEL System.

### Disposable Set: Extracorporeal Tubing Circuit

By way of example only, and not meant to be limiting, a dual stage apheresis system (the Trima System, available from CaridianBCT, Inc., Lakewood, Colo., USA) is described below. Further descriptions of the duel stage may be found in U.S. Pat. No. 6,200,287 herein incorporated by reference. It should be noted that a single stage apheresis system (the Trima Accel System, also available from CaridianBCT, Inc. Lakewood, Colo., USA) may also be used to carry out the present invention without departing from the spirit and scope of the invention. Exemplary descriptions of the single stage system may be found in U.S. Pat. Nos. 6,053,856 and 7,549,956 herein incorporated by reference.

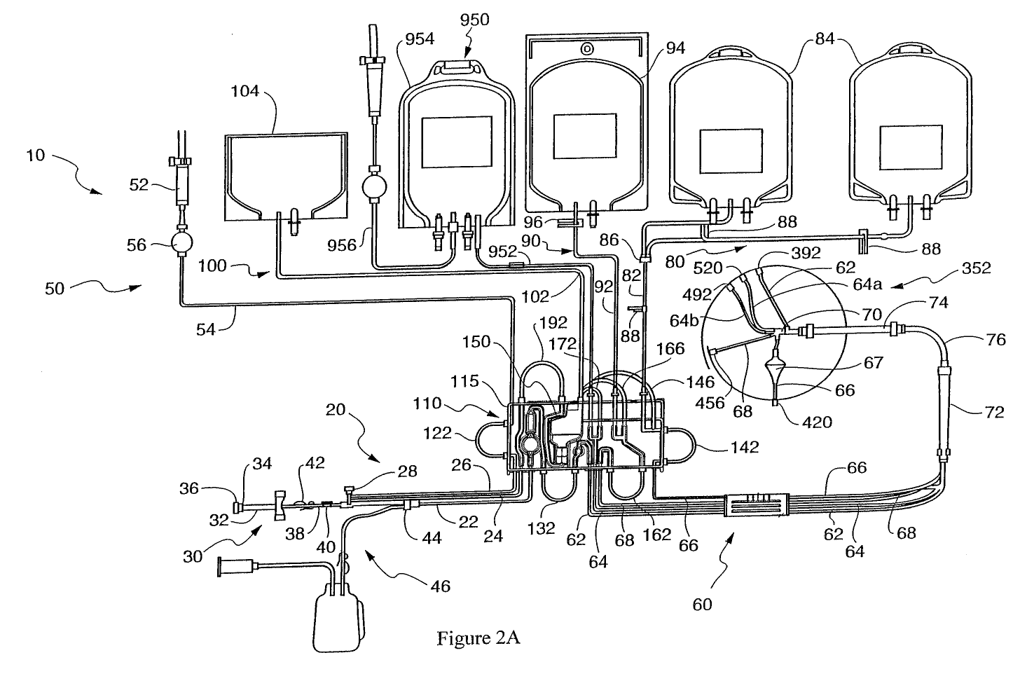

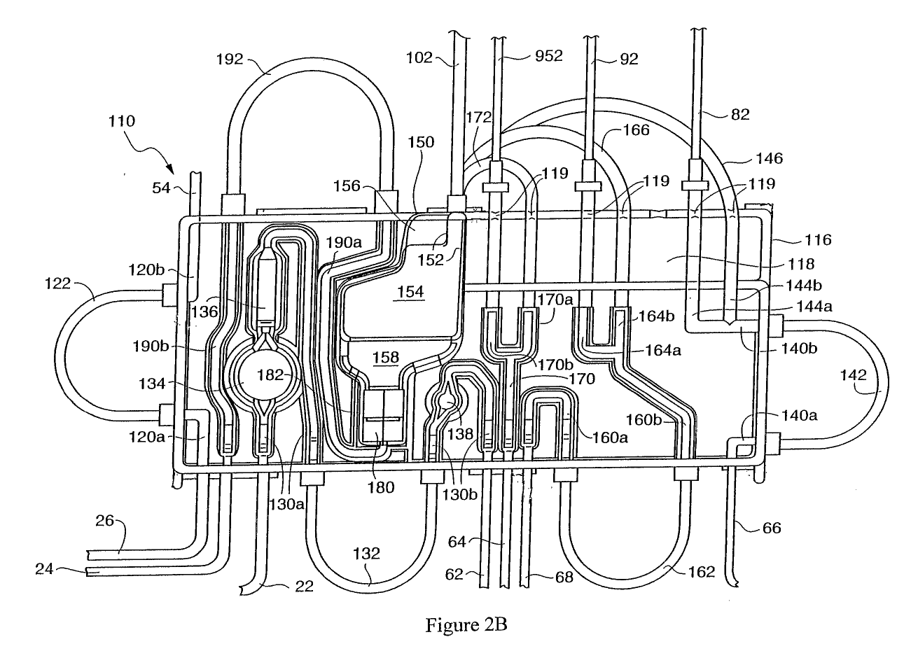

As illustrated in FIGS. 2A-2B, blood-primable extracorporeal tubing circuit 10 comprises a cassette assembly 110 and a number of tubing assemblies 20, 50, 60, 80, 90, 100 interconnected therewith. Generally, blood removal/return tubing assembly 20 provides a single needle interface between a donor/patient 4 and cassette assembly 110, and blood inlet/blood component tubing subassembly 60 provides the interface between cassette assembly 110 and blood processing vessel 352. An anticoagulant tubing assembly 50, platelet collection tubing assembly 80, plasma collection tubing assembly 90, red blood cell collection assembly 950 and vent bag tubing subassembly 100 are also interconnected with cassette assembly 110. As will be appreciated, the extracorporeal tubing circuit 10 and blood processing vessel 352 are interconnected to combinatively yield a closed disposable for a single use.

The blood removal/return tubing assembly 20 includes a needle subassembly 30 interconnected with blood removal tubing 22, blood return tubing 24 and anticoagulant tubing 26 via a common manifold 28. The needle subassembly 30 includes a needle 32 having a protective needle sleeve 34 and needle cap 36, and interconnect tubing 38 between needle 32 and manifold 28. Needle subassembly 30 further includes a D sleeve 40 and tubing clamp 42 positioned about the interconnect tubing 38. Blood removal tubing 22 may be provided with a Y-connector 44 interconnected with a blood sampling subassembly 46.

Cassette assembly 110 includes front and back molded plastic plates (not shown) that are hot-welded together to define a rectangular cassette member 115 having integral fluid passageways. The cassette assembly 110 further includes a number of outwardly extending tubing loops interconnecting various integral passageways. The integral passageways are also interconnected to the various tubing assemblies.

Specifically, cassette assembly 110 includes a first integral anticoagulant passageway 120a interconnected with the anticoagulant tubing 26 of the blood removal/return tubing assembly 20. The cassette assembly 110 further includes a second integral anticoagulant passageway 120b and a pump-engaging, anticoagulant tubing loop 122 between the first and second integral anticoagulant passageways 120a, 120b. The second integral anticoagulant passageway 120b is interconnected with anticoagulant tubing assembly 50. The anticoagulant tubing assembly 50 includes a spike drip chamber 52 connectable to an anticoagulant source, anticoagulant feed tubing 54 and a sterilizing filter 56. During use, the anticoagulant tubing assembly 50 supplies anticoagulant to the blood removed from a donor/patient 4 to reduce or prevent any clotting in the extracorporeal tubing circuit 10.

Cassette assembly 110 also includes a first integral blood inlet passageway 130a interconnected with blood removal tubing 22 of the blood removal/return tubing assembly 20. The cassette assembly 110 further includes a second integral blood inlet passageway 130b and a pump-engaging, blood inlet tubing loop 132 between the first and second integral blood inlet passageways 130a, 130b. The first integral blood inlet passageway 130a includes a first pressure-sensing module 134 and inlet filter 136, and the second integral blood inlet passageway 130b includes a second pressure-sensing module 138. The second integral blood inlet passageway 130b is interconnected with blood inlet tubing 62 of the blood inlet/blood component tubing assembly 60.

Blood inlet tubing 62 is also interconnected with input port 392 of blood processing vessel 352 to provide whole blood thereto for processing. To return separated blood components to cassette assembly 110, the blood inlet/blood component tubing assembly 60 further includes red blood cell (RBC)/plasma outlet tubing 64, platelet outlet tubing 66 and plasma outlet tubing 68 interconnected with corresponding outlet ports 492 and 520, 456, and 420 of blood processing vessel 352. The RBC/plasma outlet tubing 64 includes a Y-connector 70 to interconnect tubing spurs 64a and 64b. The blood inlet tubing 62, RBC/plasma outlet tubing 64, plasma outlet tubing 68 and platelet outlet tubing 66 all pass through first and second strain relief members 72 and 74 and a braided bearing member 76 therebetween.

Platelet outlet tubing 66 of the blood input/blood component tubing assembly 60 includes a cuvette 65 (not shown) for use in the detection of red blood cells (via an interfacing RBC spillover detector provided on blood component separation device 6) and interconnects with a first integral platelet passageway 140a of cassette assembly 110. Platelet outlet tubing 66 also includes a chamber 67, positioned in close proximity to platelet collect port 420 of blood processing vessel 352. As will be described in more detail below, during operation a saturated bed of platelets will form within chamber 67 to retain white blood cells contaminating the separated platelets within chamber 67.

The cassette assembly 110 further includes a pump-engaging, platelet tubing loop 142 interconnecting the first integral platelet passageway 140a and a second integral platelet passageway 140b. The second integral platelet passageway 140b includes first and second spurs 144a and 144b, respectively. The first spur 144a is interconnected with platelet collection tubing assembly 80.

The platelet collection tubing assembly 80 can receive separated platelets during operation and includes platelet collector tubing 82 and platelet collection bags 84 interconnected thereto via a Y-connector 86. Slide clamps 88 are provided on platelet collector tubing 82.

The second spur 144b of the second integral platelet passageway 140b is interconnected with platelet return tubing loop 146 of the cassette assembly 110 to return separated platelets to a donor/patient 4. For such purpose, platelet return tubing loop 146 is interconnected to the top of a blood return reservoir 150 integrally formed by the molded front and back plates of cassette member 115. One or more types of uncollected blood components, collectively referred to as return blood, will cyclically accumulate in and be removed from reservoir 150 during use. Back plate 114 (not shown) of the cassette member 115 also includes an integral frame corner 116 defining a window 118 through a corner of cassette member 115. The frame corner 116 includes keyhole recesses 119 for receiving and orienting the platelet collector tubing 82 and platelet return tubing loop 146 in a predetermined spaced relationship within window 118.

The plasma outlet tubing 68 of blood inlet/blood component tubing assembly 60 interconnects with a first integral plasma passageway 160a of cassette assembly 110. Cassette assembly 110 further includes a pump-engaging, plasma tubing loop 162 interconnecting the first integral plasma passageway 160a and a second integral plasma passageway 160b. The second integral plasma passageway 160b includes first and second spurs 164a and 164b. The first spur 164a is interconnected to the plasma collection tubing assembly 90.

The plasma collection tubing assembly 90 may be employed to collect it, plasma during use and includes plasma collector tubing 92 and plasma collection bag 94. A slide clamp 96 is provided on plasma collector tubing 92.

The second spur 164b of the second integral plasma passageway 160b is interconnected to a plasma return tubing loop 166 to return plasma to donor/patient 4. For such purpose, the plasma return tubing loop 166 is interconnected to the top of the blood return reservoir 150 of the cassette assembly 110. Again, keyhole recesses 119 in the frame 116 of cassette assembly 110 are utilized to maintain the plasma collector tubing 92 and plasma return tubing loop 166 in a predetermined spaced relationship within window 118.

The RBC/plasma outlet tubing 64 of the blood inlet/blood component tubing assembly 60 is interconnected with integral RBC/plasma passageway 170 of cassette assembly 110. The integral RBC/plasma passageway 170 includes first and second spurs 170a and 170b, respectively. The first spur 170a is interconnected with RBC/plasma return tubing loop 172 to return separated RBC/plasma to a donor/patient 4. For such purpose, the RBC/plasma return tubing loop 172 is interconnected to the top of blood return reservoir 150 of the cassette assembly 110. The second spur 170b may be closed off, or may be connected with an RBC/plasma collection tubing assembly 950 for collecting RBC/plasma during use. RBC collection tubing assembly 950 includes RBC collector tubing 952, an RBC collection reservoir, or bag 954, and sterile barrier filter/drip spike assembly 956. The RBC/plasma return tubing loop 172 and RBC/plasma collector tubing 952 is maintained in a desired orientation within window 118 by keyhole recesses 119 of the frame 116.

Vent bag tubing assembly 100 is also interconnected to the top of blood return reservoir 150 of cassette assembly 110. The vent bag tubing assembly 100 includes vent tubing 102 and a vent bag 104. During use, sterile air present since packaging within cassette assembly 110, and particularly within blood return reservoir 150, cyclically passes into and back out of vent tubing 102 and vent bag 104.

The platelet return tubing loop 146, plasma return tubing loop 166 and RBC/plasma return tubing loop 172 are interconnected in a row to the top of blood return reservoir 150 immediately adjacent to forwardly projecting sidewalls 152 so that the blood components returned thereby will flow down the inner walls of the blood return reservoir 150. The blood return reservoir 150 includes an enlarged, forwardly projecting mid-section 154, a reduced top section 156 and reduced bottom section 158. A filter 180 is disposed in a bottom cylindrical outlet 182 of the blood return reservoir 150.

A first integral blood return passageway 190a is interconnected to the outlet 182 of blood return reservoir 150, and is further interconnected to a second integral blood return passageway 190b via a pump-engaging, blood return tubing loop 192. The second integral blood return passageway 190b is interconnected with the blood return tubing 24 of the blood removal/return tubing assembly 20 to return blood to the donor/patient 4 via needle assembly 30.

Pump-engaging tubing loops 122, 132, 142, 162 and 192 extend from cassette member 115 to yield an asymmetric arrangement thereby facilitating proper mounting of cassette assembly 110 on blood component separation device 6 for use.

### Channel Housing

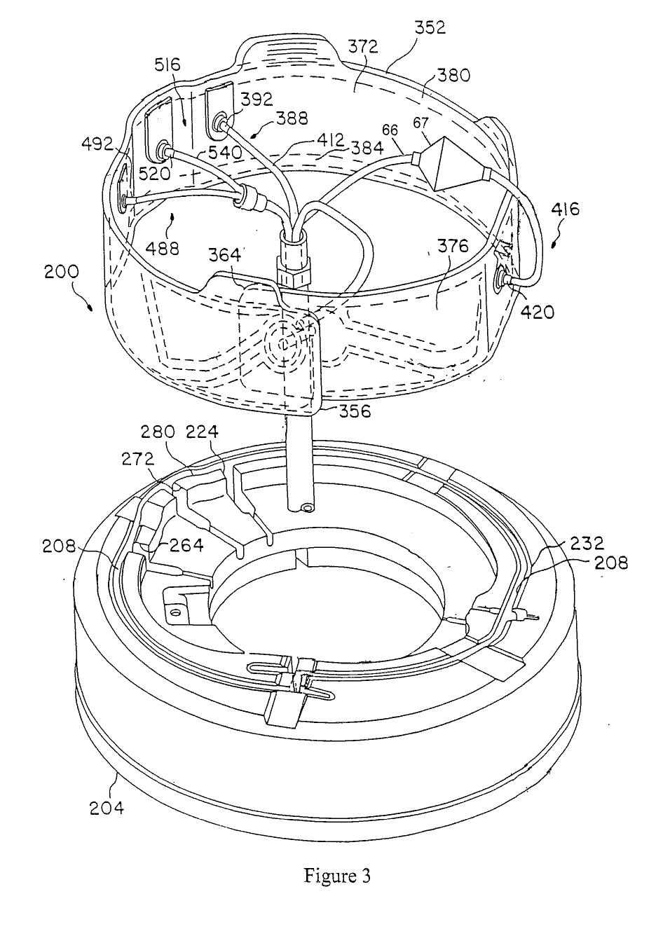

The channel assembly 200 is illustrated in FIG. 3 and includes a channel housing 204 which is disposed on the rotatable centrifuge rotor assembly 568 (FIG. 1) and which receives a disposable blood processing vessel 352.

The blood processing vessel 352 is disposed within the channel 208. Generally, the channel 208 allows blood to be provided to the blood processing vessel 352 during rotation of the channel housing 204, to be separated into its various blood component types by centrifugation, and to have various blood component types removed from the blood processing vessel 352 during rotation of the channel housing 204.

As illustrated in FIG. 3, a RBC dam 232 in the channel 208 is disposed in a clockwise direction from the blood inlet slot 224 and whose function is to preclude RBCs and other large cells such as WBCs from flowing in a clockwise direction beyond the RBC dam 232. At least in that portion of the channel 208 between the blood inlet port 224 and the RBC dam 232, blood is separated into a plurality of layers of blood component types including, from the radially outermost layer to the radially innermost layer, red blood cells (“RBCs”), white blood cells (“WBCs”), platelets, and plasma. The majority of the separated RBCs are removed from the channel 208 through an RBC outlet port assembly 516 which is disposed in an RBC outlet slot 272 associated with the channel 208, although at least some RBCs may be removed from the channel 208 through a control port assembly 488 which is disposed in a control port slot 264 associated with the channel 208.

As shown in FIG. 4, separated RBCs and other large cells as noted above are removed from the first stage 312 utilizing the above-noted configuration of the outer channel wall 216 which induces the RBCs and other large cells as noted to flow in a counterclockwise direction (e.g., generally opposite to the flow of blood through the first stage 312). Specifically, separated RBCs and other large cells as noted, flow through the first stage 312 along the outer channel wall 216, past the blood inlet slot 224 and the corresponding blood inlet port assembly 388 on the blood processing vessel 352, and to an RBC outlet slot 272. In order to reduce the potential for counterclockwise flows other than separated RBCs being provided to the control port assembly 488 disposed in the control port slot 264 a control port dam 280 of the channel 208 is disposed between the blood inlet slot 224 and the RBC outlet slot 272. That is, preferably neither WBCs nor any portion of a buffy coat, disposed radially adjacent to the separated RBCs, is allowed to flow beyond the control port dam 280 and to the control port slot 264. The “buffy coat” includes primarily WBCs, lymphocytes, and the radially outwardmost portion of the platelet layer. As such, substantially only the separated RBCs and plasma are removed from the channel 208 via the RBC control slot 264.

### Disposable Set: Blood Processing Vessel

As shown in more detail in FIG. 3, the blood processing vessel 352 is disposed within the channel 208 of channel assembly 204 for directly interfacing with and receiving a flow of blood in an apheresis procedure. The use of the blood processing vessel 352 alleviates the need for sterilization of the channel assembly 204 after each apheresis procedure and the vessel 352 may be discarded to provide a disposable system.

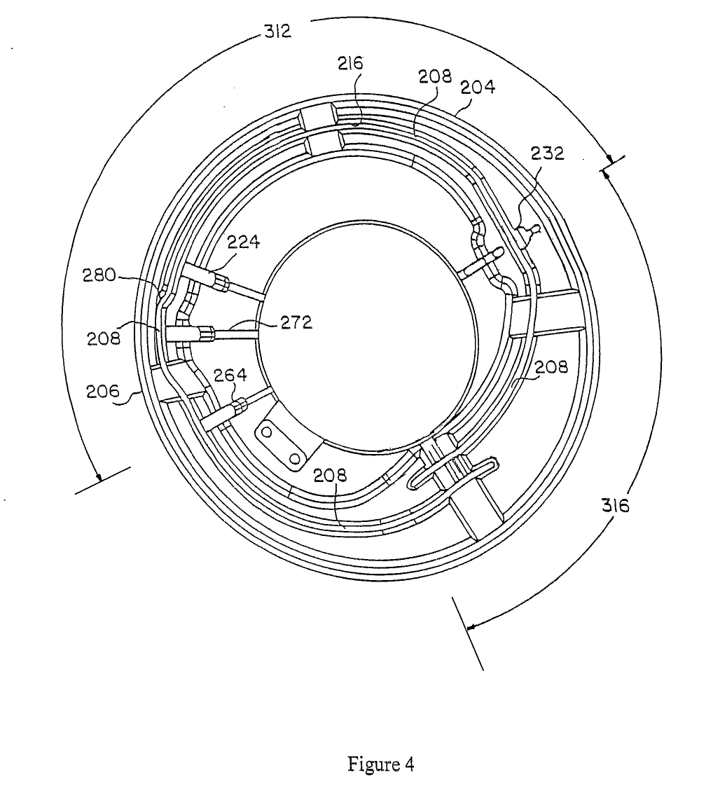

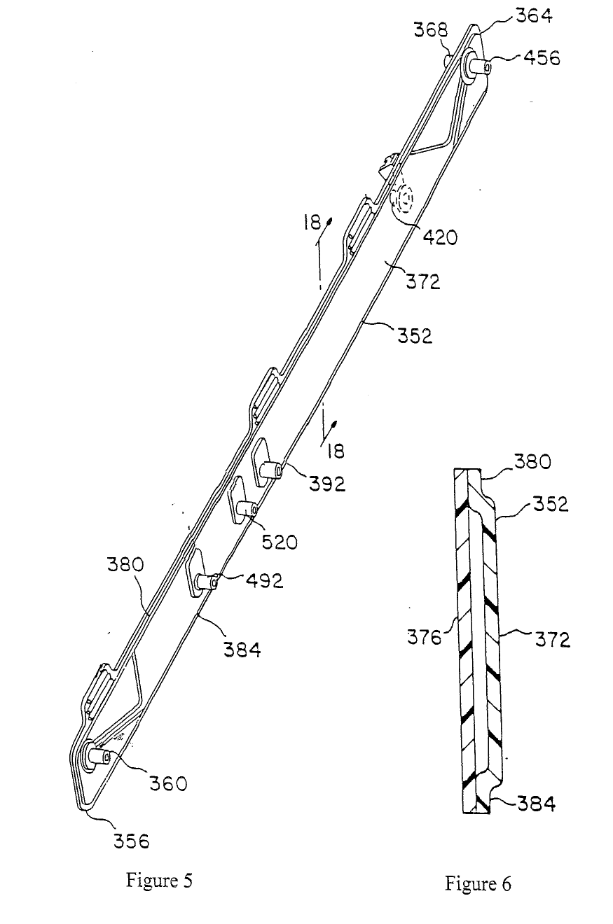

Referring primarily to FIG. 5, the blood processing vessel 352 includes a first end 356 and a second end 364 which overlaps with the first end 356 and is radially spaced therefrom. A first connector 360 is disposed proximate the first end 356 and a second connector 368 is disposed proximate the second end 364. When the first connector 360 and second connector 368 are engaged (typically permanently), a continuous flow path is available through the blood processing vessel 352.

The blood processing vessel 352 includes an inner sidewall 372 and an outer sidewall 376. In the embodiment illustrated in FIG. 6, the blood processing vessel 352 is formed by sealing two pieces of material together (e.g., RF welding). More specifically, the inner sidewall 372 and outer sidewall 376 are connected along the entire length of the blood processing vessel 352 to define an upper seal 380 and a lower seal 384. Seals are also provided on the ends of the vessel 352.

Blood is introduced into the interior of the blood processing vessel 352 through a blood inlet port assembly 388. The blood inlet port assembly 388 includes a blood inlet port 392 and a blood inlet tube 412 which is fluidly interconnected therewith exteriorly of the blood processing vessel 352. The blood inlet port 392 extends through and beyond the inner sidewall 372 of the blood processing vessel 352 into an interior portion of the blood processing vessel 352.

Separated RBCs flow along the outer sidewall 376 of the blood processing vessel 352 adjacent the outer channel wall 216, past the blood inlet port 392, and to the RBC outlet port assembly 516.

The RBC outlet port assembly 516 generally includes an RBC outlet port 520 and an RBC outlet tube 540 fluidly interconnected therewith exteriorly of the blood processing vessel 352. The RBC outlet port 520 extends through and beyond the inner sidewall 372 of the blood processing vessel 352 into an interior portion of the blood processing vessel 352.

Separated platelets are allowed to flow beyond the RBC dam 232 and into the second stage 316 (see FIG. 4) of the channel 208 in platelet-rich plasma. The blood processing vessel 352 includes a platelet collect port assembly 416 to continually remove these platelets from the vessel 352 throughout an apheresis procedure. Generally, the platelet collect port assembly 416 is disposed in a clockwise direction from the blood inlet port assembly 388, as well as from the RBC dam 232 when the blood processing vessel 352 is loaded into the channel 208. Moreover, the platelet collect port assembly 416 interfaces with the outer sidewall 376 of the blood processing vessel 352.

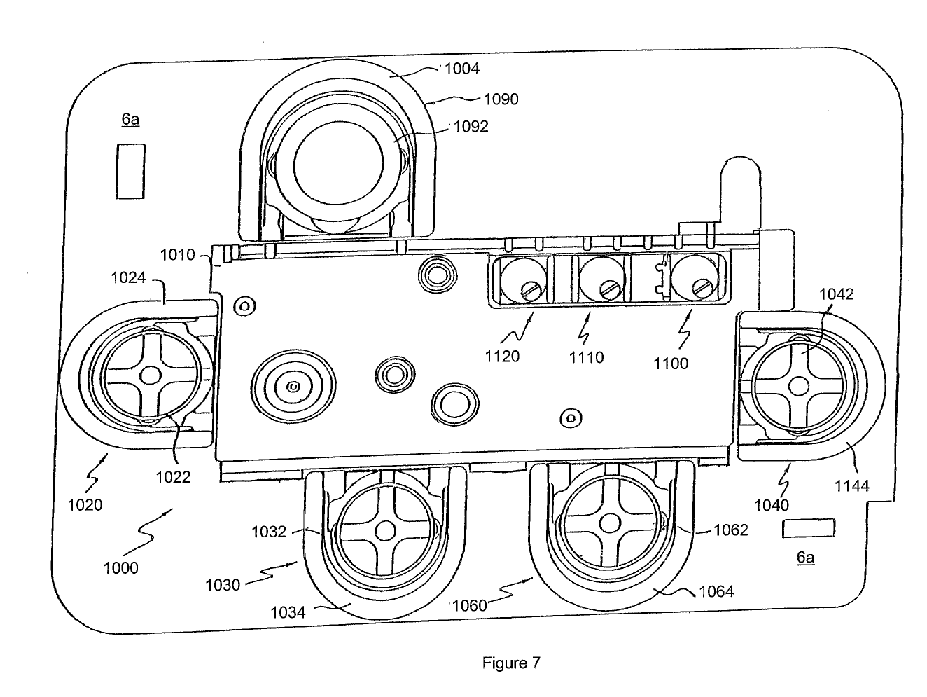

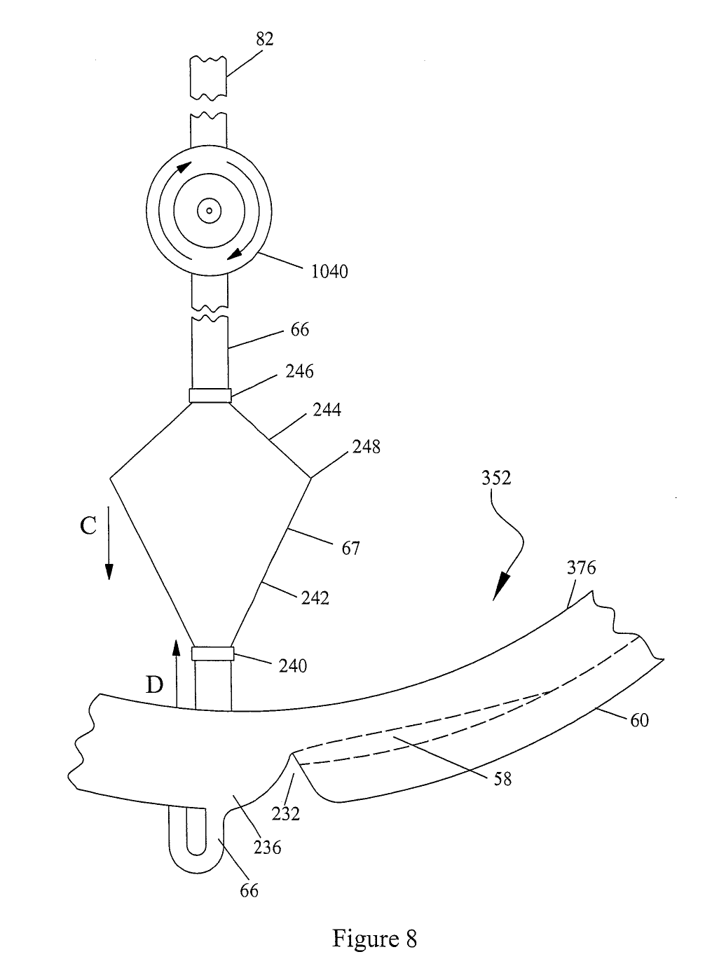

The platelet collect port assembly 416 generally includes a platelet collect port 420 and a platelet collect tube 66 (see FIG. 8) which is fluidly interconnected with fluid chamber 67.

As illustrated in FIG. 8, the fluid chamber 67 is formed by joining a first chamber section 242 having inlet 240 to a second chamber section 244 having outlet 3246.

The volume of the fluid chamber 67 should be at least large enough to accommodate enough platelets to provide a saturated fluidized particle bed (described below) for a particular range of flow rates, particle sizes and centrifuge rotor 200 speeds.

The fluid chamber interior has a maximum cross-sectional area 248 located at a position intermediate the inlet 240 and outlet 246 where sections 242, 244 join. The cross-sectional area of the fluid chamber interior decreases, or tapers from the maximum cross-sectional area 248 as shown in FIG. 8. Although the fluid chamber 67 is depicted with two sections 242, 244 having frustoconical interior shapes, the interior shapes may be paraboloidal, or of any other shape having a major cross-sectional area greater than the inlet or outlet area. The fluid chamber 67 may be constructed from a unitary piece of plastic rather than from separate sections.

### Pump/Valve/Sensor Assembly

As noted, cassette assembly 110 is mounted upon and operatively interfaces with the pump/valve/sensor assembly 1000 of blood component separation device 6 during use. The pump/valve/sensor assembly 1000 as illustrated in FIG. 7 includes a cassette mounting plate 1010, and a number of peristaltic pump assemblies, flow divert valve assemblies, pressure sensors and ultrasonic level sensors interconnected to face plate 6a of blood collection device 6 for pumping, controlling and monitoring the flow of blood/blood components through extracorporeal tubing circuit 10 during use.

More particularly, anticoagulant pump assembly 1020 is provided to receive anticoagulant tubing loop 122, blood inlet pump assembly 1030 is provided to receive blood inlet tubing loop 132, platelet pump assembly 1040 is provided to receive platelet tubing loop 142, plasma pump assembly 1060 is provided to receive plasma tubing loop 162, and blood return pump assembly 1090 is provided to receive blood return tubing loop 192.

Each of the peristaltic pump assemblies 1020, 1030, 1040, 1060, and 1090 includes a rotor 1022, 1032, 1042, 1062 and 1092, and raceway 1024, 1034, 1044, 1064, and 1094 between which the corresponding tubing loop is positioned to control the passage and flow rate of the corresponding fluid.

Platelet divert valve assembly 1100 is provided to receive platelet collector tubing 82 and platelet return tubing loop 146, plasma divert valve assembly 1110 is provided to receive plasma collector tubing 92 and plasma return tubing loop 166, and RBC/plasma divert valve assembly 1120 is provided to receive RBC/plasma return tubing loop 172 and RBC/plasma collector tubing 952.

### Operation of Extracorporeal Tubing Circuit and Pump/Valve/Sensor Assembly

In an initial blood prime mode of operation, blood return pump 1090 is operated in reverse to transfer whole blood through blood removal/return tubing assembly 20, integral blood return passageway 190, blood return tubing loop 192 and into reservoir 150. Contemporaneously and/or prior to the reverse operation of blood return pump 1090, anticoagulant peristaltic pump 1020 is operated to prime and otherwise provide anticoagulant from anticoagulant tubing assembly 50, through anticoagulant integral passageways 120a, 120b and into blood removal tubing 22 and blood return tubing 24 via manifold 28.

In the blood processing mode, the blood inlet peristaltic pump 1030, platelet peristaltic pump 1040 and plasma peristaltic pump 1060 are operated continuously.

In normal operation, whole blood will pass through needle assembly 30, blood removal tubing 22, cassette assembly 110 and blood inlet tubing 62 to processing vessel 352. The whole blood will then be separated in vessel 352. A separated platelet stream will pass out of port 420 of the vessel, through platelet tubing 66, through chamber 67, back through cassette assembly 110, through tubing 82 to be collected in collector assembly 80. Similarly, separated plasma will exit vessel 352 through port 456 to plasma tubing 68 back through cassette assembly 110, and will then either be collected in plasma tubing assembly 90 or diverted to reservoir 150. Further, separated red blood cells and plasma may pass through ports 492 and 520 of vessel 352 through RBC/plasma tubing 64, through cassette assembly 110 and either into reservoir 150 or into RBC/plasma collector tubing assembly 950 for collection.

### Platelet Collection and Purification

The blood separation control device 6 provides control signals to pump/valve/sensor assembly 1000 so that platelet divert valve assembly 1100 diverts the flow of separated platelets pumped through platelet outlet tubing 66 into chamber 67 for further purification and finally into platelet collection tubing 82 for collection in bag 84.

To separate contaminating white blood cells from platelets in chamber 67, plasma, the least dense blood component, flows within the separation vessel 352 along the top surface of the buffy coat layer 58 (see FIG. 8). When the height of the buffy coat layer 58 approaches the top of RBC dam 232, the flowing plasma washes the platelets and some white blood cells of the buffy coat layer 58 over the RBC dam 232 into the platelet collect well 236.

Plasma then carries platelets and white blood cells from the platelet collect well 236 (see FIG. 8) into the fluid chamber 67, which is filled with a priming fluid, so that a saturated fluidized particle bed may be formed. The processor maintains the rotation speed of the rotor 200 within a predetermined rotational speed range to facilitate formation of this saturated fluidized bed. In addition, the processor regulates platelet pump 1040 to convey plasma, platelets, and white blood cells at a predetermined flow rate through the tubing segment 66 and into inlet 28 of the fluid chamber 67. These flowing blood components displace the priming fluid from the fluid chamber 67.

When the platelet and white blood cell particles enter the fluid chamber 67, they are subjected to two opposing forces. Plasma flowing through the fluid chamber with the aid of pump 1040 establishes a first viscous drag force when plasma flowing through the fluid chamber 67 urges the particles toward the outlet 32 in the direction “D”, shown in FIG. 7. A second centrifugal force created by rotation of the rotor 200 and fluid chamber 67 acts in the direction “C” to urge the particles toward the inlet 28.

The processor regulates the rotational speed of the rotor 200 and the flow rate of the pump 1040 to collect platelets and white blood cells in the fluid chamber 67. As plasma flows through the fluid chamber 67, the flow velocity of the plasma decreases as the plasma flow approaches the maximum cross-sectional area 33. This flow reaches a minimum velocity at this maximum cross-sectional area 33. Because the rotating centrifuge rotor 200 creates a sufficient gravitational field in the fluid chamber 67, the platelets accumulate near the maximum cross-sectional area 33 rather than flowing from the fluid chamber 67 with the plasma. The white blood cells accumulate somewhat below the maximum cross-sectional area 33. However, density inversion tends to mix these particles slightly during this initial establishment of the saturated fluidized particle bed.

The larger white blood cells accumulate closer to the chamber inlet 28 than the smaller platelet cells, because of their different sedimentation velocities. Preferably, the rotational speed and flow rate are controlled so that very few platelets and white blood cells flow from the fluid chamber 67 during formation of the saturated fluidized particle bed.

The platelets and white blood cells continue to accumulate in the fluid chamber 67 while plasma flows through the fluid chamber 67. As the concentration of platelets increases, the interstices between the particles become reduced and the viscous drag force from the plasma flow gradually increases. Eventually the platelet bed becomes a saturated fluidized particle bed within the fluid chamber 67. Since the bed is now saturated with platelets, for each new platelet that enters the saturated bed in the fluid chamber 67, a single platelet must exit the bed. Thus, the bed operates at a steady state condition with platelets exiting the bed at a rate equal to the rate additional platelets enter the bed after flowing through inlet 28.

Although the bed is saturated with platelets, a small number of white blood cells may be interspersed in the platelet bed. These white blood cells, however will tend to “fall” or settle out of the platelet bed toward inlet 28 due to their higher sedimentation velocity. Most white blood cells generally collect within the fluid chamber 67 between the saturated platelet bed and the inlet 28. Thus, the bed effectively filters white blood cells from the blood components continuously entering the fluid chamber 67, while allowing plasma and platelets released from the saturated bed to exit the chamber 67.

However, because cells in chamber 67 are separated based on size, the white cells having a smaller than average size may escape the platelet bed and contaminate the concentrated platelets. Although the number of WBCs which escape the chamber 67 are small, (an average of less than 10⁵ rWBC end up in the platelet product, compared to 5×10⁹ being retained within the chamber 67) as discussed in the background above, even this small number of WBC can cause undesirable side effects in the recipient of the platelet product.

As discussed above, after further purification of the platelets in chamber 67, the purified platelets flow out of chamber 67, through tubing 82 and into platelet collect bag(s) 84. The purified platelets in bag(s) 84 may then be resuspended in a liquid which enables the platelets to be stored over time. Resuspension/storage liquid could be added directly to the platelets contained in bag 84, or the platelets could be transferred out of bag 84 into a bag containing the resuspension/storage fluid. Resuspension/storage fluid could also be in bag 84 before the platelets are added.

One fluid which may be used as a resuspension fluid/platelet storage solution is Isolyte S (available from B Braun). Isolyte S is commonly used as an intravenous electrolyte solution. This multi-electrolyte injection solution has a well-characterized safety profile in the United States, and contains ingredients known to support platelet storage. 100 mL of Isolyte S pH 7.4 contains 0.53 g sodium chloride, 0.5 g sodium gluconate, 0.37 g sodium acetate trihydrate, 0.037 g potassium chloride and 0.03 g magnesium chloride hexahydrate and is made by processing the constituents in water. The solution has an osmolarity of around 295 mOsm. Isolyte S is not commonly used as a platelet additive solution. However, when used as an additive solution for platelet storage, it appears to have the added benefit of disintegrating contaminating white blood cells which escaped the saturated fluidized particle bed within chamber 67 and are contained in apheresed platelets.

As discussed above, it is undesirable to have even a small amount of white blood cells contaminating a platelet concentrate, whether they escaped through a leukoreduction filter or a saturated fluidized particle bed within chamber 67. The below experiments demonstrate that if Isolyte S is used as a platelet additive solution, residual white blood cells contaminating an apheresed platelet concentrate are disintegrated.

### Results

### Example 1

A single hyperconcentrated platelet product was collected on the TRIMA apheresis machine 6, purified in chamber 67, and stored in platelet bag 84. The content of platelet bag 84 was divided into 7 small (50 mL) bags. Plasma or the following constituents were added to each small bag (the platelets included a 37.5% plasma carryover): saline, saline+Mg²⁺, SSP⁺, Isolyte S, Isolyte S+Citrate and Isolyte S−Mg²⁺. The concentrations of Mg²⁺ or citrate added were comparable to what is found in current PAS solutions (see Table 1 below). The primary differences between Isolyte S and the below listed PAS solutions are that Isolyte S lacks citrate and contains twice the amount of magnesium. Note: Plasmalyte A has the same constituents as Isolyte S, it merely has a different manufacturer.

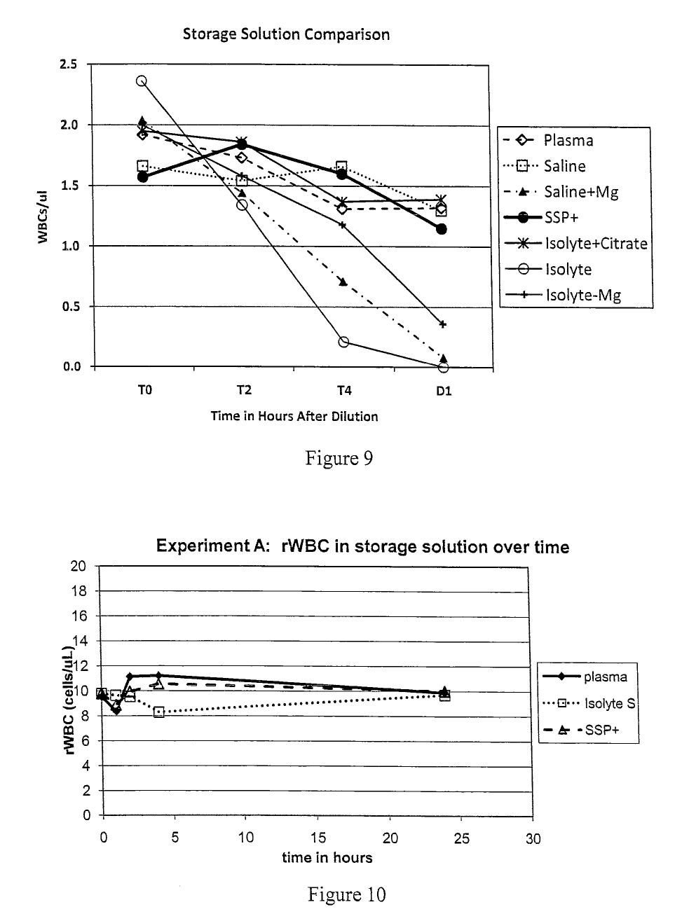

Residual WBC (rWBC) samples were taken and measured at approximately 2 hour intervals for 4 hours and then again on Day 1 (after overnight storage on a flatbed rotator). The initial (T0) time point was taken immediately after mixing. The results are shown in FIG. 9.

As seen in FIG. 9:
rWBC counts decrease by ˜30% by Day 1 in plasma.
rWBC counts decrease by ˜30% by Day 1 in SSP⁺.
rWBC counts decrease by <30% by Day 1 in saline.
rWBC counts decrease by ˜100% by Day 1 in Isolyte S.
rWBC counts decrease by ˜100% by Day 1 in saline+Mg²⁺.
rWBC counts decrease by ˜30% by Day 1 in Isolyte S+citrate.
rWBC counts decrease by ˜80% by Day 1 in Isolyte S−Mg²⁺.

Although rWBC decrease as a function of time regardless of the storage medium, rWBC counts decreased at a much faster rate when stored in a medium containing a moderate amount of magnesium compared to plasma or other platelet additive solutions.

Using Isolyte S as a platelet additive solution may help to reduce the number of residual WBC in apheresed platelet products.

The above experiment appears to suggest that the lack of citrate and inclusion of approximately at least a moderate amount of Mg²⁺ in Isolyte S as compared to the amount present in the other solutions in Table 1 may be responsible for the observed decrease in rWBC counts in Isolyte S-stored hyperconcentrated platelets collected using apheresis.

TABLE 1
Concentration of Electrolytes

| | | | PAS-2 | PAS-3 | | PAS-3M |
|---|---|---|---|---|---|---|
| (mEq/L) | Plasmalyte A | Isolyte S | T-sol | Intersol | Composol | SSP+ |
| Sodium | 140 | 141 | 176 | 191.9 | 173 | 183.9 |
| Potassium | 5 | 5 | — | — | 5 | 5 |
| Magnesium | 3 | 3 | — | — | 1.5 | 1.5 |
| Chloride | 98 | 98 | 116 | 77.3 | 98 | 77.2 |
| Acetate | 27 | 27 | 30 | 32.5 | 27 | 32.5 |
| Gluconate | 23 | 23 | — | — | 23 | — |
| Phosphate | — | 1 | — | 28.2 | — | 28.2 |
| Citrate | — | — | 10 | 10.8 | 11 | 10.8 |
| pH | 7.4 | 7.4 | 7.2 | 7.2 | 7.0 | 7.2 |

### Example 2

This study looked at the effects of platelet additive solution containing moderate amounts of magnesium on residual white blood cells contained in hyperconcentrated platelet products. Hyperconcentrated platelet products are platelets which are collected at a high enough concentration that they require dilution in a storage solution. A concentration greater than or equal to 2100×10³/μl is considered a hyperconcentrated platelet product. Because of the lack of plasma in a hyperconcentrated platelet product, platelet quality degrades after 48 hours of storage. Therefore, hyperconcentrated platelets must be diluted in a platelet additive solution to allow for seven days of storage.

Table 2 shows rWBC data for paired hyperconcentrated platelet products collected on the Trima Accel System. The first column represents rWBC counts for individual platelet products before addition of a platelet storage solution containing moderate amounts of magnesium; the second column represents rWBC counts for individual platelet products after addition of a platelet additive solution containing moderate amounts of magnesium. The data in Table 2 demonstrates that the rWBC concentration decreases by 70% on average with the addition of platelet additive solution containing moderate amounts of magnesium. Decrease in rWBC concentration indicates that WBCs are being disintegrated by a platelet additive solution containing moderate amounts of magnesium.

TABLE 2
rWBC concentration data for paired platelet products.

| Pre-Isolyte | Post-Isolyte | Change in |
|---|---|---|
| rWBC | rWBC | rWBC |
| WBC/uL | WBC/uL | % |
| 3.36 | 1^{†} | 70% |
| 3.42 | 1.82 | 47% |
| 3.84 | 1^{†} | 74% |
| 3.99 | 1^{†} | 75% |
| 4.59 | 1^{†} | 78% |
| 5.67 | 1^{†} | 82% |
| 5.76 | 1^{†} | 83% |
| 7.95 | 1^{†} | 87% |
| 42.09 | 20.51 | 51% |
| 192.36 | 93.5 | 51% |
| | Avg | 70% |

^{†}Value of 1 represents the lower detection limit of the assay

### Example 3

One hypothesis for the unexpected results using an additive solution containing moderate amounts of magnesium as a platelet additive solution is that the population of WBC that escapes the saturated fluidized particle bed in chamber 67 or a leukoreduction filter media, are not representative of the WBC population as a whole. In order to escape the saturated fluidized particle bed in the chamber 67 and leukoreduction media, this subpopulation of WBC may be smaller and less dense than the average WBC. Internal studies have shown that WBCs carried over in standard platelet products are enriched in B lymphocytes. 39% of the WBCs found in platelets apheresed using a Trima apheresis machine are B lymphocytes, as compared to the 3.3% found in whole blood. Moreover, lymphocytes make up 28% of WBC in whole blood but make up 98% of WBC found in platelets separated using the Trima apheresis system. This smaller, denser subpopulation of WBCs from apheresed blood may be more sensitive to the residual amount of Ca⁺² remaining (originating from the initial whole blood) in the apheresed platelets in combination with the Mg⁺² ions found in an additive solution containing moderate amounts of magnesium, and therefore more susceptible to apoptosis or lysis.

To determine whether the rWBCs contaminating the platelet concentrates differ from normal lymphocytes in their response to exposure to an additive solution containing moderate amounts of magnesium, mononuclear cells were isolated from a whole blood buffy coat preparation using Leukocyte Separation Medium (LSM® Lymphocyte Separation Medium commercially available from MP Biomedicals, Solon, Ohio, USA). The buffy coats were separated from whole blood using an automated whole blood separation system (the Atreus System, available from CaridianBCT, Inc., Lakewood, Colo.). These cells are considered to be normal, unselected mononuclear cells. The cells were then suspended at a concentration of 10/μl in anticoagulated plasma and in anticoagulated plasma plus additive solution at a ratio of 1:2, using SSP+ and Isolyte S as the additive solutions. As seen in FIG. 10, over 24 hours there was no significant decrease in WBC concentration as determined by flow cytometry in the plasma or in either of the plasma-additive solution products. “Normal” mononuclear cells, then, in plasma: additive solution containing moderate amounts of magnesium at a volume ratio of 1:2, differ from rWBC in the platelet concentrates in that they do not show the rapid decreases in concentration seen with the rWBC from apheresed platelets.

In the BD Leucocount (commercially available kit from Becton Dickinson, Franklin Lakes, N.J., USA) flow cytometric assay the rWBCs are labeled with propidium iodide (PI), a DNA dye. In order to allow the PI to enter the nuclei of the leukocytes, the sample preparation includes treatment with detergent (EDTA) to make the cell and nuclear membranes permeable. The Leucocount assay was carried out on platelet products in the presence and absence of detergent. In theory, only WBCs whose membranes were compromised or “leaky” would be stained by PI in the absence of detergent treatment. The percent of WBC with permeable membranes was calculated according to:

$$\frac{W B C s stained by PI without detergent}{W B C s stained using the standard assay ( with detergent )} \times 100$$

Two controls were tested; in one, WBCs had been stored for 19 days in EDTA tubes and were seriously compromised by the detergent. For this sample, 100% of WBCs stained by the standard assay were stained in the absence of detergent. In comparison, only 61% of WBCs stored in EDTA for two days were stained. Ninety two percent (92%) of the rWBCs from apheresis platelet products collected on the Trima Accel system were stained in the absence of detergent treatment, while only 3% of WBCs that were isolated from the saturated fluidized particle bed in chamber 67 in the same collection were stained. This is shown in FIG. 11. Those WBCs isolated from chamber 67 had not “escaped” to become rWBCs, and were representative of the “normal” population of retained leukocytes.

The lack of effect of platelet additive solution containing moderate amounts of magnesium on “normal” WBCs and the demonstration that the rWBC in platelet concentrates collected on the Trima Accel System have permeable membranes, supports the hypothesis that the rWBCs in platelet concentrates collected on the Trima and Trima Accel Systems represent a subpopulation of leukocytes that are compromised, perhaps in the early stages of apoptosis, and may be susceptible to rapid disintegration in the presence of mediators, such as ionized magnesium.

### Example 4

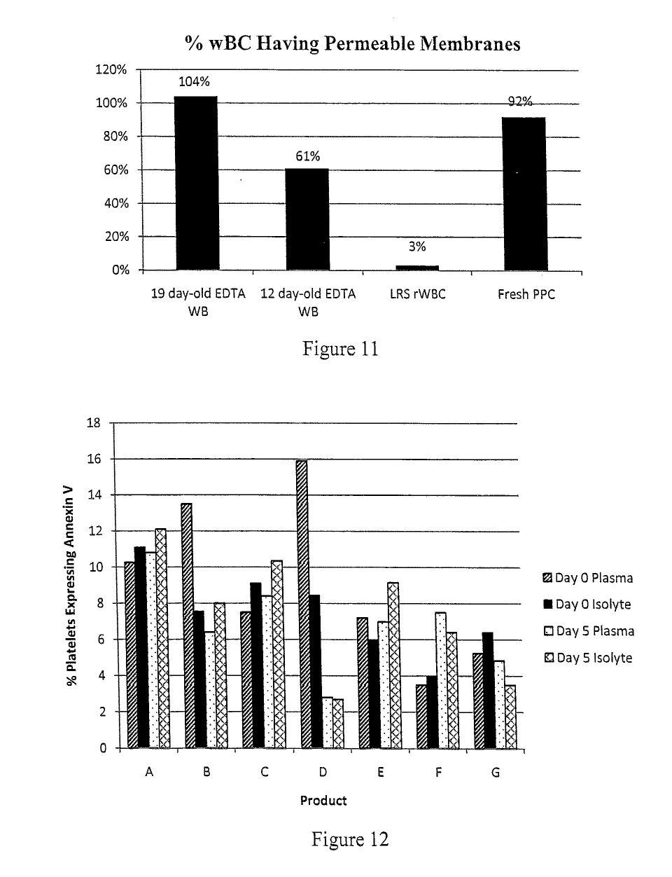

If the rWBC stored in an additive solution containing moderate amount of magnesium with 35% plasma carryover undergo accelerated apoptosis due to the presence of moderate concentrations of ionized magnesium, the question can arise as to whether this additive/storage medium has similar effects on the platelets. This is answered, in part, by the observation that the platelet concentrations are maintained throughout the storage period, and that in vitro platelet quality data of the additive solution containing moderate amounts of magnesium-stored platelets differ little from those stored in plasma alone. To investigate this further, the differences in Annexin V expression in paired platelet products stored in plasma and in additive solution containing moderate amounts of magnesium with 35% plasma carryover were studied. Annexin V binds to phosphatidyl serine exposed on the outside of cell membranes. It detects membrane changes that are early signs of apoptosis in leukocytes and platelets. FIG. 12 shows the results of seven paired studies that demonstrate no significant differences in Annexin V expression between plasma-stored and 35% plasma carryover-additive solution containing moderate amounts of magnesium-stored platelets. This observation shows that the storage conditions do not induce measurable apoptosis in the platelets.

### Example 5

This study focused on providing in vitro quality data to support additive solution containing moderate amounts of magnesium as a platelet additive solution.

This was a paired study in which subjects donated platelet products by apheresis on the Trima Accel system. Each subject underwent a Control, standard single platelet unit collection (standard platelet collected on Trima Accel and stored in plasma) and a Test, single unit collection (hyperconcentrated platelet collected on Trima Accel in significantly less plasma, diluted and stored in additive solution containing moderate amount of magnesium) on the same day, separated by at least 90 minutes. In other words, Test platelets were collected in a hyperconcentrated state and diluted with additive solution containing moderate amount of magnesium prior to storage, whereas Control platelets are collected and stored at standard concentration.

The order of Test and Control collections was randomized. The Control platelet units mirrored the Test platelet units in terms of yield and final concentration.

The hyperconcentrated platelet (Test) products were resuspended in additive solution containing moderate amounts of magnesium to a plasma carryover (=plasma volume/[plasma volume+P.A.S. volume]) of 35%. Plasma volume collected by apheresis includes a minor amount (16-20% of total volume) of ACD-A, which is the anticoagulant mixed with donor blood entering the Trima disposable tubing set during an apheresis procedure. Therefore, the actual plasma volume at 35% plasma carryover is 28-29% of the volume of the stored platelet concentrate. All platelet products were stored at 20-24° C. with agitation for seven days. Samples were taken from the stored platelet products on days 1, 5, and 7 of storage.

The primary outcome for this study was that 95% or more of the Test units have Day 5 pH at 22° C. greater than 6.2 with one-sided confidence limit of 95% (0/60 failures) in accordance with the FDA Guidance for Industry and FDA Review Staff: Collection of Platelets by Automated Methods, December 2007. This is shown in Table 3.

TABLE 3

| Test | | Control |
|---|---|---|
| Average | 7.4 | 7.5 |
| St Dev | 0.2 | 0.1 |
| Min | 6.7 | 7.2 |
| Max | 7.6 | 7.7 |
| N | 67 | 67 |

Table 4 represents the average values of other measures of in vitro platelet quality during storage of Test and Control platelets. p-selectin, ESC, HSR, and morphology were measured after 5 Day storage. These values are within expected ranges for both Test and Control platelet products.

| | | | |
|---|---|---|---|
| P-selectin | ESC | HSR | Morphology |

Test

| | | | | |
|---|---|---|---|---|
| Average | 22.8 | 23.2 | 53.2 | 290 |
| St Dev | 15.4 | 4.9 | 12.4 | 49 |
| Min | 1.7 | 14.2 | 16.5 | 200 |
| Max | 72.0 | 39.1 | 72.0 | 385 |
| N | 67 | 67 | 67 | 67 |

Control

| | | | | |
|---|---|---|---|---|
| Average | 15.1 | 24.9 | 55.6 | 293 |
| St Dev | 9.7 | 6.0 | 10.9 | 48 |
| Min | 2.0 | 11.8 | 29.5 | 205 |
| Max | 52.0 | 44.4 | 77.0 | 388 |
| N | 67 | 67 | 67 | 67 |

The results of these investigations of the effects of 35% plasma carryover-additive solution containing moderate amounts of magnesium-storage on the rWBCs contained in platelet concentrates collected on the Trima Accel System strongly suggest that the rWBCs consist primarily of membrane permeable, possibly early apoptotic, lymphocytes. The low citrate concentration in the storage medium, coupled with moderate concentrations of magnesium, a known mediator of lymphocyte apoptosis, results in rapid disintegration of the initial low numbers of contaminating leukocytes. The rapid disappearance of intact leukocytes in platelet concentrates collected on the Trima Accel System and stored in 35% plasma carryover and additive solution containing moderate amounts of magnesium has no detectable effects on the platelets. The plasma-additive solution containing moderate amounts of magnesium does not induce apoptosis in the platelets or degradation in platelet quality over the period of storage.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention.

1. A method of reducing residual white blood cells in an apheresed platelet product comprising the step of:
adding to the platelet product a solution comprising sodium chloride and magnesium; and
inducing degradation of the residual white blood cells
whereby the degradation of the residual white blood cells caused by the addition of the solution reduces any residual white blood cells which may be contained in the platelet product.

2. The method of claim 1 wherein the solution further comprises gluconate, acetate tryhydrate, and potassium.

3. The method of claim 1 wherein the solution lacks citrate.

4. The method of claim 1 further comprising leukoreducing the platelet product prior to the adding step.

5. The method of claim 4 wherein the leukoreducing step comprises leukoreducing by forming a saturated fluidized particle bed.

6. The method of claim 3 wherein the leukoreducing step comprises leukoreducing by passing the platelet product through leukoreducing media.

7. The method of claim 1 further comprising maintaining the quality of the platelet product.

8. The method of claim 2 further comprising preparing the solution by the process of formulating in water per 100 mL of solution: 0.53 g sodium chloride, 0.5 g sodium gluconate, 0.37 g sodium acetate tryhydrate, 0.037 g potassium chloride and 0.03 g magnesium chloride hexahydrate.

9. The method of claim 8 wherein the solution has a pH of 7.4 and an osmolarity of around 295 mOs.

10. A platelet storage solution for reducing the amount of residual white blood cells in apheresed platelets comprising sodium chloride and magnesium and lacking citrate.

11. The storage solution of claim 10 further comprising gluconate, acetate tryhydrate, and potassium.

12. The storage solution of claim 11 wherein the storage solution is prepared by the process of formulating in water per 100 mL of solution: 0.53 g sodium chloride, 0.5 g sodium gluconate, 0.37 g sodium acetate tryhydrate, 0.037 g potassium chloride and 0.03 g magnesium chloride hexahydrate.

13. The storage solution of claim 12 wherein the pH is 7.4 and has an osmolarity of around 295 mOs.

14. A method for further leukoreducing leukoreduced apheresed platelets suitable for transfusion into a patient, the method comprising:
providing hyperconcentrated apheresed leukoreduced platelets and
resuspending the platelets in a solution containing at least sodium chloride and magnesium and lacking citrate.

15. The method of claim 14 wherein the solution further comprises gluconate, acetate tryhydrate, and potassium.
